(19) United States
(12) Patent Application Publication
(10) Pub. No.: US 2025/0107887 A1
(43) Pub. Date: Apr. 3, 2025

(54) INJECTOR FOR AN INTRAOCULAR LENS, AND SYSTEM HAVING AN INJECTOR

(71) Applicant: IOLUTION GmbH, Hamburg (DE)

(72) Inventors: Christoph MAROSCHECK, Hamburg (DE); Maximiliano GUTIERREZ, Hamburg (DE)

(73) Assignee: IOLUTION GmbH, Hamburg (DE)

(21) Appl. No.: 18/684,370

(22) PCT Filed: Aug. 11, 2022
(86) PCT No.: PCT/EP2022/072505

§ 371 Date: Feb. 16, 2024

(30) Foreign Application Priority Data
Aug. 19, 2021 (DE) 10 2021 121 575.7

(51) Int. Cl. A61F 2/16

(52) U.S. Cl. CPC A61F 2/1678; A61F 2/1667; A61F 2/1691

(57) ABSTRACT

The disclosure relates to an injector for intraocular lenses. The injector may comprise an automatically activatable haptics slider. Furthermore, the injector can be configured such that a folding member for folding the intraocular lens is blocked when the haptics slider has not been activated. The disclosure furthermore relates to a system comprising a container in which an intraocular lens is stored in a cartridge. The haptics slider becomes automatically activated when the cartridge is received by the injector housing.

### CROSS-REFERENCE TO RELATED APPLICATION

This application is a national stage application, filed under 35 U.S.C. § 371, of International Patent Application PCT/EP2022/072505, filed on Aug. 11, 2022, which claims the benefit of German Patent Application DE 10 2021 121 575.7, filed on Aug. 19, 2021.

### TECHNICAL FIELD

The disclosure relates to an injector for an intraocular lens. Furthermore, the disclosure relates to a system comprising a container for storing and transporting an intraocular lens and an injector for the intraocular lens.

### BACKGROUND

Injectors have been known for applying an artificially manufactured intraocular lens, for example as part of a cataract operation.

Usually, such injector is a tool in the form of a disposable instrument which includes a plunger that can be used by the surgeon to push the lens out of a cannula and into the eye.

The intraocular lenses made of a plastics material can be folded for application. This allows for the passage from which the lens is pushed out to have a smaller cross section.

Furthermore, such lenses usually have what is known as haptics. These haptics are springy unfoldable arms which center the lens in the patient's eye. Advantageously, the haptics are folded inward before folding the lens.

For this purpose, published patent application WO 2018/006889 A2 (Iolution GmbH) discloses a system comprising an injector and a cartridge that can be slid onto the injector and which includes the lens. The cartridge comprises a haptics slider which, according to the teaching of the above-mentioned document, comprises two laterally arranged sliding members, by means of which the user first folds inward the haptics before then closing a lid having a folding blade for folding the lens. The lens will now be in a folded state with the arms folded inward and can then be applied by the surgeon.

### SUMMARY

The disclosure provides an injector and a system comprising an injector for an intraocular lens, in which actuation of the slider for the haptics of the intraocular lens is further simplified.

This is achieved by an injector for an intraocular lens and by a system comprising a container and comprising an injector for an intraocular lens according to any one of the independent claims.

Preferred embodiments and refinements of the invention will be apparent from the subject-matter specified in the dependent claims, the description and the drawings.

According to a first aspect, the invention relates to an injector for an intraocular lens.

The injector comprises a housing with a plunger that is slideable inside the housing for applying the intraocular lens. The plunger is connected to a handle which the user can use to advance the plunger, in one embodiment also via a spindle drive, so as to inject the lens into the eye through a cannula.

The housing may consist of a plurality of parts. More particularly, the housing may comprise a first part which comprises the cannula for applying the lens and which can be coupled with a cartridge in which the intraocular lens is disposed. A second housing part may comprise a guide for the plunger and the plunger rod.

The injector preferably comes in the form of a disposable instrument made of plastics material. The components of the injector are preferably coupled to each other without the use of an adhesive. More particularly, the housing parts can be snap-connected to each other.

The injector comprises a haptics slider which can be activated by slidingly fitting the cartridge onto the housing of the injector.

The haptics slider may be automatically activated by sliding the cartridge onto the injector.

More particularly it is contemplated that the cartridge is slid on from the distal side of the injector in the proximal direction, i.e. with respect to the central axis of the plunger.

According to another embodiment, however, it is also possible for the cartridge to be slid onto the housing of the injector from above, from a lateral side, or from below.

The injector is in particular designed such that the folding member can only be enabled after the haptics have been folded inward using the haptics slider.

The haptics slider preferably forms part of the cartridge.

It is in particular suggested for the haptics slider to comprise a first sliding member and a second sliding member.

In particular, the sliding members may be arranged in a plane above or below the plane in which the intraocular lens is supported inside the cartridge.

The sliding members may in particular be in the form of laterally displaceable slides which move in opposite directions and each of which folds inward a respective arm of the haptics.

Preferably, the sliding members are coupled to each other, in particular through a rocker which is coupled to the sliding members in an articulated manner.

One of the sliding members or a component coupled to the sliding member is moved when the cartridge is slidingly fitted, namely by abutting on a stop in the housing of the injector, i.e. by being held in place.

The opposite sliding member can be moved in the other direction through a pivotably mounted rocker.

The haptics will thus be folded inward from both sides towards the optical portion of the intraocular lens, in a simple manner.

According to a further aspect, the disclosure relates to an injector for an intraocular lens, in particular to an injector as described above.

The injector comprises a housing with a plunger that is slideable inside the housing for applying the intraocular lens. The injector moreover comprises a slide-on cartridge for the intraocular lens.

The injector also comprises a haptics slider for folding inward the haptics of the intraocular lens.

Furthermore, the injector comprises a folding member for folding the intraocular lens.

As described above in the introductory part, the folding member is used to fold the intraocular lens so that it becomes more compact and better fits through the cannula.

The folding member may in particular be provided on a flap which is folded onto the cartridge. When being folded, the folding member will plunge into the cartridge, thereby folding the intraocular lens and transferring it from the cartridge into a passage through which the lens can be pushed out of the injector by the plunger.

The folding member will only be enabled for folding the intraocular lens after the haptics slider has been actuated.

This means that the folding member can only fulfill its intended function when the haptics of the intraocular lens have already been folded inward using the haptics slider.

In the non-actuated state of the haptics slider, by contrast, the folding member will be blocked or inhibited such that it cannot plunge into the cartridge and transfer the intraocular lens into the housing of the injector.

According to one embodiment, it is the haptics slider or a component coupled to the haptics slider, which blocks the folding member.

In particular, the haptics slider, as long as it has not been actuated, can serve as a stop for the folding member which is in the form of a flap.

According to one embodiment, the haptics slider comes in the form of a turntable which provides a passage for the folding member once it has been rotated into the actuated position.

In the non-actuated state, the passage for the folding member will be aligned transversely to the folding member. This will block the folding member from plunging into the cartridge.

A further aspect of the disclosure relates to an injector for an intraocular lens, in particular to one of the injectors described above.

This injector comprises a housing with a plunger that is slideable inside the housing for applying the intraocular lens, a slide-on cartridge for the intraocular lens, a folding member for folding the intraocular lens, and a haptics slider for folding inward the haptics of the intraocular lens.

The haptics slider comprises a first and a second sliding member, the first and second sliding members being configured so as to move in the same direction when the haptics are folded inward.

In contrast to the prior art described above, according to which the sliding members move in different directions, only a single direction of movement is suggested in this embodiment.

For example, this may be a rotation around an axis.

The haptics slider may in particular be configured so as to be rotatable. More particularly, the haptics slider is defined by a turntable.

On the one hand, this configuration makes it easier to actuate the haptics slider if the latter is operated manually.

Furthermore, the haptics slider can easily be provided in the form of a blocking element which blocks the plunging of the folding blade in the non-actuated state.

Once the haptics are in an inward folded position, however, the haptics slider will provide a passage for the folding member.

According to a further aspect, the disclosure relates to an injector for an intraocular lens, in particular an injector as described above.

This injector comprises a housing with a plunger that is slideable inside the housing for applying the intraocular lens, a cartridge for the intraocular lens that can be slid onto the housing of the injector, a folding member for folding the intraocular lens, and a haptics slider for folding inward the haptics of the intraocular lens.

The haptics slider comprises a first and a second sliding member, and the first and second sliding members can be moved towards each other in order to fold inward the haptics.

In contrast to the prior art described in the introductory part, according to which the sliding members move parallel to each other and in different directions, it is therefore suggested for the sliding members to move towards each other.

If the haptics slider is operated manually, this makes it easier to activate the haptics slider. For example, the cartridge can in particular be gripped with two fingers. When the two sliding members are pressed together, they will move towards each other and fold inward the opposite arms of the haptics.

This may in particular be implemented by sliding members that are arranged laterally, i.e. on the left and right of the housing.

According to a further embodiment, the sliding members are moved towards each other in the axial direction in terms of the central axis of the plunger.

It is in particular contemplated for the injector, in particular for the cartridge of the injector, to be configured of a telescopic kind. In this case, the intraocular lens can be carried in an intermediate part. A distal sliding member folds inwards one arm of the haptics of the intraocular lens. The intermediate part slides into a proximal sliding member, or the proximal sliding member is displaced relative to the distal sliding member due to the sliding insertion of the intermediate part and folds inwards an opposite arm of the haptics.

This embodiment of the invention is particularly suitable for an automated inward folding of the haptics.

In particular, the distal sliding member can be captured when the cartridge is slidably fitted. When the cartridge is slid onto the housing of the injector, the cartridge is coupled with the housing of the injector and simultaneously pushed together such that the distal and proximal sliding members are moved towards each other.

Furthermore, automated activation of the haptics slider is also possible by having the distal sliding member abutting against a stop of a container, for example in a water container in which the cartridge containing the intraocular lens is stored, when the housing of the injector is introduced into the container in order to couple the cartridge to the housing.

A further aspect of the disclosure relates to an injector for an intraocular lens, in particular an injector as described above.

The injector comprises a housing with a plunger that is slideable inside the housing for applying the intraocular lens, a cartridge for or including the intraocular lens, which can be slid onto the housing, a folding member for folding the intraocular lens, and a haptics slider for inward folding of the haptics of the intraocular lens.

The haptics slider is arranged on an underside of the cartridge.

Underside of the cartridge refers to that side which adjoins the housing of the injector when the cartridge is used as intended. The folding member, on the other hand, may plunge through the top of the cartridge.

The arrangement on the underside, in particular in a plane which lies below the plane in which the intraocular lens is carried in the cartridge, provides for a particularly tamper-resistant design of the haptics slider, on the one hand.

In this embodiment, the haptics slider may in particular comprise two sliding members that are arranged laterally opposite each other.

These sliding members are moved in opposite directions towards each other in order to fold inward the arms of the haptics.

According to a preferred embodiment, only one of the sliding members is moved relative to the housing. In this embodiment, the lens itself is moved by the first sliding member. More particularly, the lens can be moved through a rotational movement towards the second sliding member which is in the form of a stop for an arm of the haptics and thus will fold inward the haptics arm which is located opposite the first sliding member.

The disclosure furthermore relates to a system comprising an injector for an intraocular lens, in particular an injector as described above.

The injector comprises a haptics slider for folding inward the haptics of the intraocular lens.

The system furthermore comprises a container.

The container may in particular be a liquid-filled container for storing and transporting a hydrophilic intraocular lens.

Furthermore, the container may be a heating container which is configured for heating the lens carried in the cartridge before its application so that it will be more flexible and can be folded more easily.

The haptics slider can be activated by introducing the injector into the container.

In the case where a heating container is used, for example, the haptics slider may be activatable by introducing the cartridge together with the injector.

Furthermore, the cartridge may be accommodated in the container in a housing portion intended for this purpose, in particular in a passage.

In this case, the system is configured such that the cartridge can be picked up by the housing of the injector.

When the cartridge is coupled to the injector, the haptics slider will automatically be activated.

This can be implemented, for example, by causing the cartridge and/or the housing of the injector to be pressed against a stop, which activates the haptics slider.

This also allows to provide for particularly easy handling of the system comprising the injector. Furthermore, as stated above, the folding member may be blocked until the haptics slider has been activated by the coupling.

The container may comprise a receptacle for the cartridge and/or for the injector. Furthermore, the container may have a stop for the cartridge and/or for the injector, which is designed so as to activate the haptics slider when the injector is introduced into the container.

In one embodiment, the cartridge is accommodated in the receptacle of the container. The haptics slider is activated when the injector is introduced into the container and is thereby coupled to the cartridge.

### BRIEF DESCRIPTION OF THE DRAWINGS

The subject-matter of the invention will be explained in more detail below with reference to various exemplary embodiments.

FIGS. 1 to 3 show a first exemplary embodiment of an injector in which the haptics slider is automatically activated when the cartridge is picked up from a container.

FIG. 4 shows the cartridge accommodated in the container.

FIGS. 5 to 7 show an embodiment of a cartridge in which the haptics slider is automatically activated when the cartridge is slid onto the housing of the injector.

FIG. 7 is a longitudinal sectional view along a horizontal plane.

FIGS. 8 to 10 show an injector in which the cartridge has a haptics slider comprising two sliding members which can be moved towards each other axially.

FIG. 11 is a schematic view of an injector comprising sliding members that can be moved towards each other axially.

FIG. 12 is a schematic view showing sliding members arranged on the lateral side of the cartridge, which can move towards each other and which at the same time serve as a grip piece.

FIGS. 13 to 18 are schematic views of an injector comprising a haptics slider in the form of a turntable.

FIGS. 19 to 21 are schematic views of an injector in which the haptics slider is located on the underside of the cartridge.

### DETAILED DESCRIPTION

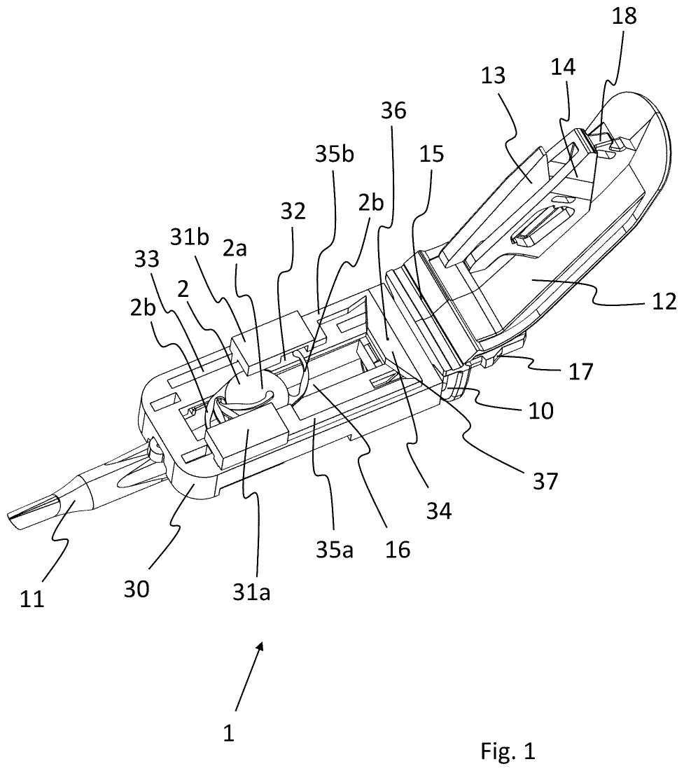

FIG. 1 is a perspective view of an injector 1 which is configured such that the haptics slider 31a, 31b is automatically activated when the cartridge 30 is picked up from a container.

The perspective view shows the housing 10 of the injector, of which only the front housing part is illustrated, which can be connected to a rear housing part through locking hooks 17.

The rear housing part including the handle, plunger rod and actuator is not shown. It can be configured in accordance with the prior art described above.

The cartridge 30 can be slid onto the housing 10.

The intraocular lens 2 is arranged inside the cartridge 30. The intraocular lens 2 comprises a lens body 2a constituting the optical portion of the lens, and the haptics 2b which in this exemplary embodiment comprise two resilient outwardly springing arms.

Housing 10 comprises a flap 12 arranged thereon. The flap includes a folding member 13 which has a blade-like shape and is movably mounted in bearing block 14.

Film hinge 15 allows the folding member 13 to be introduced into the cartridge 30 thereby folding the intraocular lens 2 and transferring it into the passage 16 of housing 10, from where the folded intraocular lens 2 can be applied out of the cannula 11 using the plunger, not shown.

Flap 12 has a locking hook 18 which will lock the flap 12 on the cartridge 30 once it has been folded down. This enhances tamper-resistance of the injector system which comes in the form of a disposable system.

In a further embodiment, the flap may also lock on at least one side wall of the cartridge (not shown).

For folding inward the haptics 2a, 2b, laterally arranged sliding members 31a, 31b are used which can be displaced relative to each other along rails which are in the form of a groove 33, for example.

The view illustrates the non-folded state.

The folding members 31a, 31b comprise a housing each having a recess 32 in which the end of the respective arm of the haptics 2b sits. By displacing sliding member 31a in a proximal direction and displacing sliding member 31b in a distal direction, the arms of haptics 2b are folded over or onto the optical portion 2a of the intraocular lens 2, before the lens 2 will then be folded up by the folding member 13 and transferred into the passage 16.

Folding members 31a, 31b are coupled to a rocker 34 via bars 35a, 35b running in the longitudinal direction.

Rocker 34 is pivotally mounted on the axis of rotation 36. Rocker 34 itself is coupled to bars 35a, 35b via hinges 37 in the form of film hinges.

As soon as the first sliding member 31a abuts on a stop, it will be shifted in the proximal direction thereby moving the rocker 34 through bar 35a and thereby, on the opposite side, moving the sliding member 31b in the distal direction through bar 35b.

Thus, it is sufficient to exert a force on one of the sliding members 31a in a single direction in order to move both sliding members 31a, 31b in opposite directions.

In a further embodiment, not shown here, the sliding members 31a and 31b are not coupled to each other. In this case, sliding member 31a can be activated, for example, when picking up the injector from a container (by pushing it forward). Sliding member 31b can be activated when the injector including the cartridge is pulled out of the container, for example by virtue of an undercut in the container. Otherwise, this embodiment may be configured as shown here in FIGS. 1 through 3.

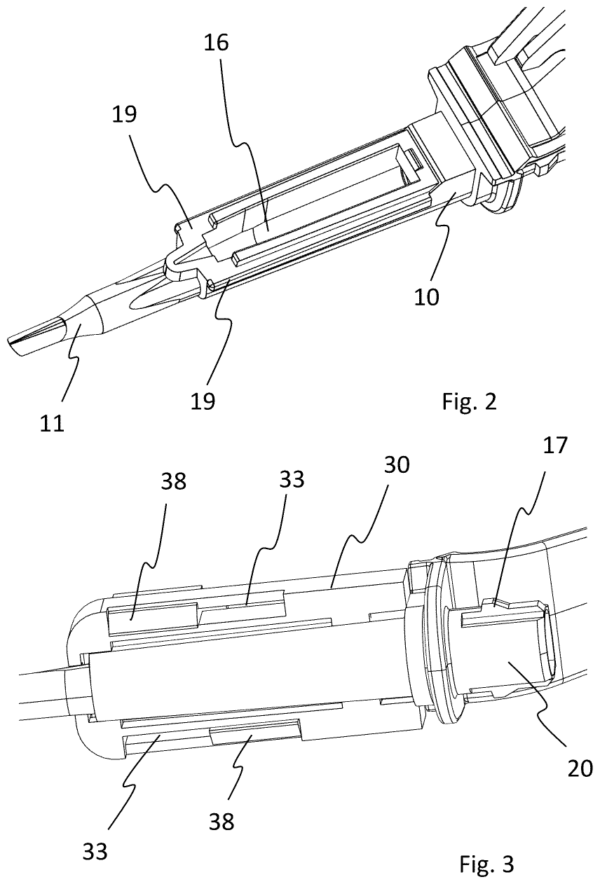

FIG. 2 illustrates the housing 10 with the cartridge removed.

A rail 19 can be seen onto which the cartridge can be slidably fitted.

Within the range of the cartridge, the housing 10 opens at the top, and passage 16 is open, through which the lens is transferred into the cannula 11.

FIG. 3 is a perspective view of the underside of the injector shown in FIGS. 1 and 2.

Cartridge 30 has again been slidably fitted here.

On the underside of cartridge 30 it can be seen that locking hooks 38 extend through the grooves 33 which are in the form of through-grooves in the housing of the cartridge 30. This allows for easy mounting of the cartridge.

Proximally of the cartridge 30, connecting piece 20 is provided which has locking hooks 17 which serve to couple the housing part illustrated here to a rear housing part.

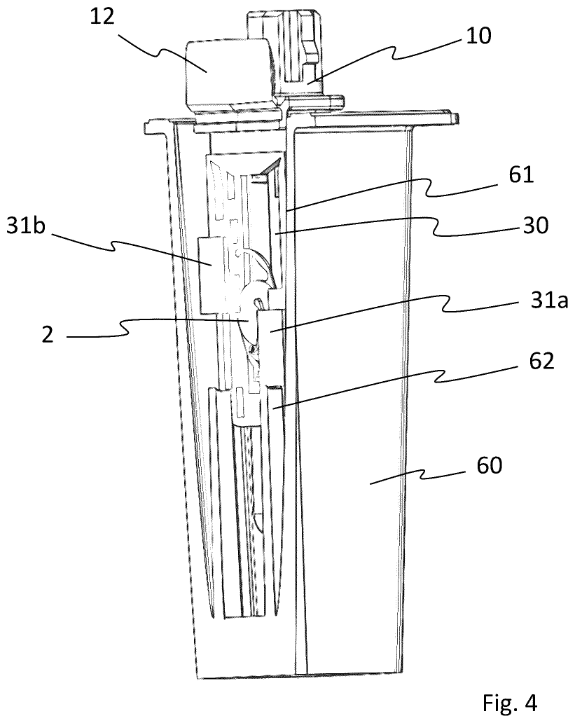

FIG. 4 schematically illustrates how the cartridge 30 is accommodated in a container 60 and is picked up by the housing 10 of the injector.

The intraocular lens 2 is preloaded inside cartridge 30.

In this exemplary embodiment, cartridge 30 is arranged in a container 60 which is filled with water. This system is preferably used for hydrophilic intraocular lenses 2.

Container 60 has a passage 61 which provides an accommodation area adapted to the cartridge 30.

After opening the container 60, the injector is immersed into the container with the housing 10 first and is coupled to the cartridge 30.

Thereby, the cartridge 30 with the first haptics slider 31a in front thereof will be urged against a stop 62.

Thus, during the coupling process, the cartridge 30 is pushed deeper into the container 60 thereby moving the haptics sliders 31a and 31b so that the arms of the haptics are folded inward onto the optical portion of the intraocular lens 2.

The cartridge 30 preferably locks inseparably with the housing 10 of the injector and can then be removed together with the intraocular lens.

The user now only needs to fold inward the flap 12 in order to transfer the intraocular lens 2 into the passage leading to the cannula.

The injector will then be ready for use and can be used immediately to apply the lens into the eye.

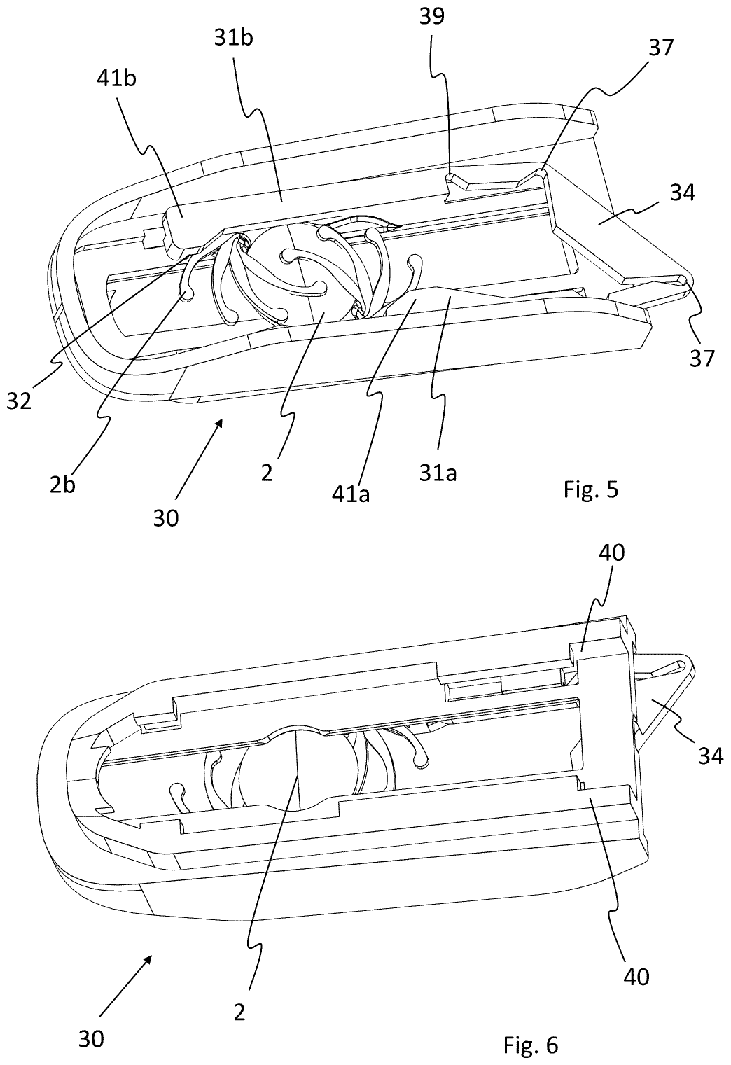

FIG. 5 is a perspective view of a cartridge 30 which is configured such that the haptics slider 31a, 31b is automatically activated when the cartridge 30 is slid onto the injector.

In this exemplary embodiment, the haptics slider comprises sliding members 31a, 31b on laterally opposite sides, which are displaceable in the longitudinal direction and which are mounted on a rail, for example a groove, as in the embodiment shown in FIGS. 1 to 4.

Furthermore, the sliding members 31a, 31b also each have a recess 32 into which the unfolded arm of the haptics 2b projects.

In this exemplary embodiment, the recesses 32 are provided in a head 41a, 41b which is enlarged compared to the adjacent portion of the respective sliding member 31a, 31b. This allows an engagement surface for the haptics 2b within the recess 32 to be aligned obliquely, which makes it easier to fold inward the haptics 2b.

Again, in this exemplary embodiment, the sliding members 31a, 31b are coupled to a rotatably mounted rocker 34.

Rocker 34 is coupled to the sliding members 31a, 31b through a hinge 37 (e.g. a film hinge).

In contrast to the previously illustrated exemplary embodiment, a further hinge 39 is provided.

The intermediate portion provided between first hinge 37 and second hinge 39 allows to compensate for the fact that the extension of the rocker 34 in the transverse direction decreases with increasing deflection of the rocker 34.

This view shows the state where the haptics slider is not activated. The movement of the haptics arms 2b is illustrated schematically by the three superimposed positions.

Cartridge 30 is configured such that the haptics slider is activated by slidingly fitting the cartridge 30 onto the housing of the injector.

In the present exemplary embodiment this is achieved by the rocker 34 being positioned transversely in the non-activated state shown here such that it protrudes from the basic housing of the cartridge 30.

When slid onto the injector housing, the rocker 34 will abut on the injector housing.

The housing of the injector thus acts as a stop which causes the rocker 34 to straighten.

Thereby, sliding member 31b will be moved in the proximal direction, and sliding member 31a will be moved in the distal direction. As a result, the arms of the haptics 2b will be folded inward towards the optical portion of lens 2.

FIG. 5 shows the top of cartridge 30. Thus, in this embodiment, again, the sliding members 31a, 31b move in a plane that is above the plane in which the lens 2 is supported inside the cartridge. Here, the haptics are driven on the haptics to optics connection end, in FIGS. 1-3 on the opposite end.

FIG. 6 is a perspective view of the underside of cartridge 30.

Here the cartridge has opposing rails 40 for being slidably fitted on the housing of the injector. The rocker 34 can be seen to protrude on the proximal side of the cartridge 30.

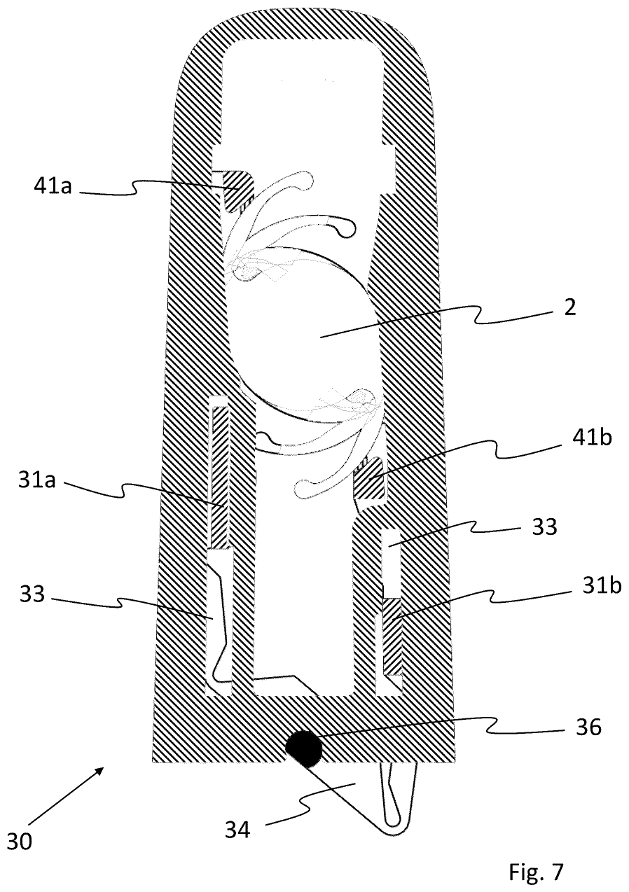

FIG. 7 is a longitudinal sectional view along a horizontal plane. It can be seen that the sliding members 31a, 31b each engage in a groove in the housing of the cartridge 30. Thus, they are guided similarly as in the embodiment shown in FIG. 3.

However, in this embodiment the sliding members 31a, 31b only engage in the grooves 33 in a proximal area of the cartridge. In the distal direction, the sliding members 31a, 31b extend on top of the cartridge up to the head 41a, 41b, which protrudes downward into the area in which the lens 2 is disposed.

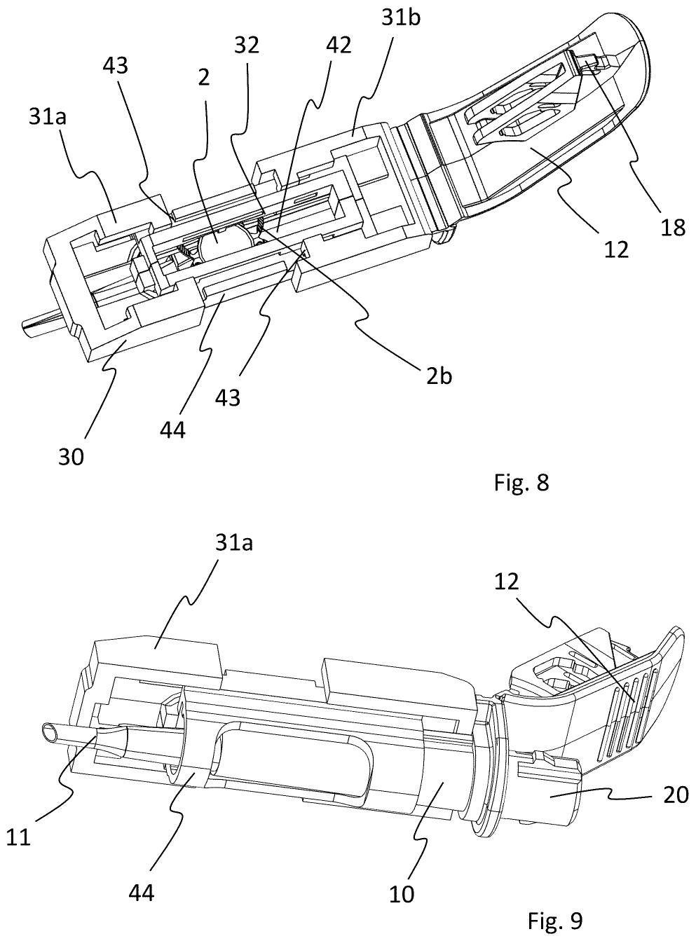

FIG. 8 is an embodiment in which the haptics slider 2 comprises opposing sliding members 31a, 31b which can be moved towards each other in the axial direction. The injector or the cartridge 30 of the injector comprises an intermediate part 44 which can be displaced relative to the sliding members 31a, 31b via a rail fit with rails 43.

What is shown is the non-activated state.

The sliding members 31a, 31b each have at least one recess 32 into which the arms 2b of the haptics protrude.

If now a force is exerted on the distal sliding member 31a (either manually or by the sliding member 31a abutting on a stop in a container, as in FIG. 4), the sliding members 31a, 31b and the intermediate part 44 will be telescoped together.

Thereby, the haptics 2b of lens 2 will be folded inward. First, the distal arm of the haptics will be folded inward by sliding member 31a. Then, the intermediate part 44 will slide in the proximal direction and the proximal arm of the haptics 2b will be folded inward when sliding member 31a abuts on sliding member 31b.

In this embodiment, the injector may be configured in such a way that in the non-activated state as shown here, the flap 12 with the folding member cannot be closed because it cannot plunge through the support plane 42 for the lens 2. This is because the intermediate part 44 has not yet been pushed in.

Only when the sliding members 31a and 31b have been pushed together the flap 12 can be closed and the locking hook 18 will lock the flap 12.

The sliding elements 31a, 31b and the intermediate part 44 will preferably lock in the fully collapsed position.

This embodiment enables the displacing of one respective haptic distally and proximally, or, depending on the design of the lens shape, of two respective haptics distally and proximally.

FIG. 9 is a perspective view of the underside of the injector.

In the non-activated state, the distal sliding member 31a projects beyond the intermediate part 44 and is located above the cannula 11.

The intermediate part 44 surrounds the cannula 11.

In this exemplary embodiment, the housing 10 is again made up of a plurality

of parts. The front housing part illustrated here comprises a connecting portion 20 for a rear housing part.

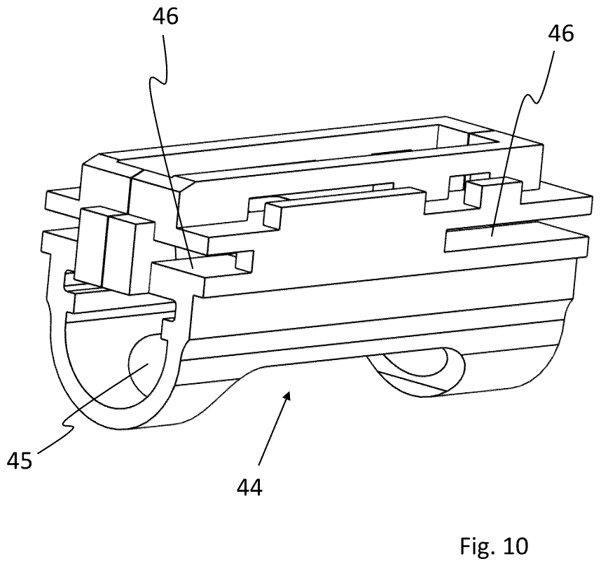

FIG. 10 is a perspective view of the intermediate part 44. It has a passage 45 through which the cannula will extend in the assembled state, which cannula includes the passage into which the folded lens is transferred.

The intermediate part 44 furthermore has rails 46 which serve to movably couple it with the sliding members.

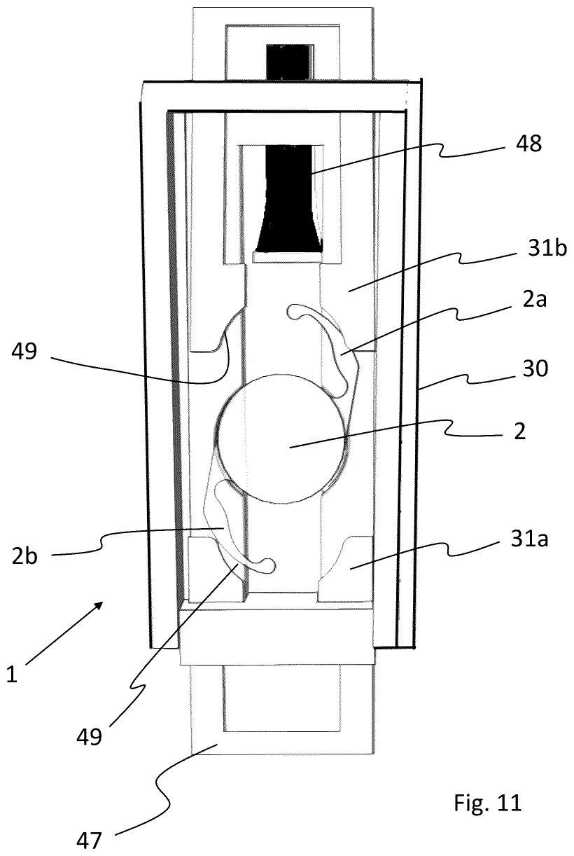

FIG. 11 is a further schematic view of an injector 1 which comprises sliding members 31a, 31b that can be moved towards each other axially.

The proximal sliding member 31b is coupled with the injector housing.

The distal sliding member 31a can be displaced in the proximal direction via front portion 47.

The sliding members 31a, 31b each have curved engagement surfaces 49 which simplify the inward folding of the haptics 2b.

The proximal sliding member 31b is displaced towards the lens 2 either manually or by sliding the cartridge 30 onto the injector housing.

Once the haptics 2b has been folded inward, a flap comprising a folding member can again be closed in this exemplary embodiment, too, thereby folding the lens and transferring it into a passage disposed below the cartridge. Then, the lens 2 can be pushed out of the cannula, which is not shown in this schematic view, using the plunger 48 which has a preferably soft end portion (for example made of silicone), and can thus be applied.

Conceivable options include a variant in which the sliding members 31a, 31b are activated manually, for example when being removed from a container, as well as a variant in which the sliding members 31a, 31b are shifted inwards when the cartridge 30 is slid onto the injector housing.

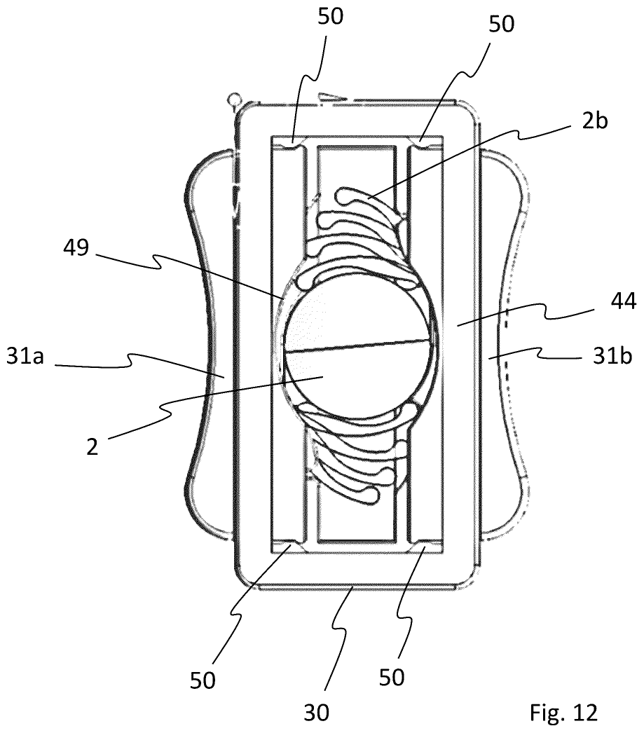

FIG. 12 is a schematic view of an exemplary embodiment in which the sliding members 31a, 31b protrude laterally from the housing of the cartridge.

For folding inward the haptics 2b, each of the sliding members 31a, 31b includes a curved engagement surface 49.

The sliding members 31a, 31b are each in the form of a grip piece and may have a curved outer contour.

What is shown is the non-activated state. The movement of the arms of the haptics 2b is again schematically illustrated by superimposed positions.

The user can grip the cartridge 30 on the lateral sliding members 31a, 31b.

If the user now presses the sliding members 31a, 31b together, they will move towards each other and will fold inward the haptics 2b.

In the inward folded state, the sliding members 31a, 31b will lock onto the housing of the cartridge 30 by virtue of locking hooks 50.

This embodiment allows for a very simple manual activation of the haptics slider. It can virtually be activated “on the fly” when the user grabs the cartridge 30 to slide it onto the housing of the injector.

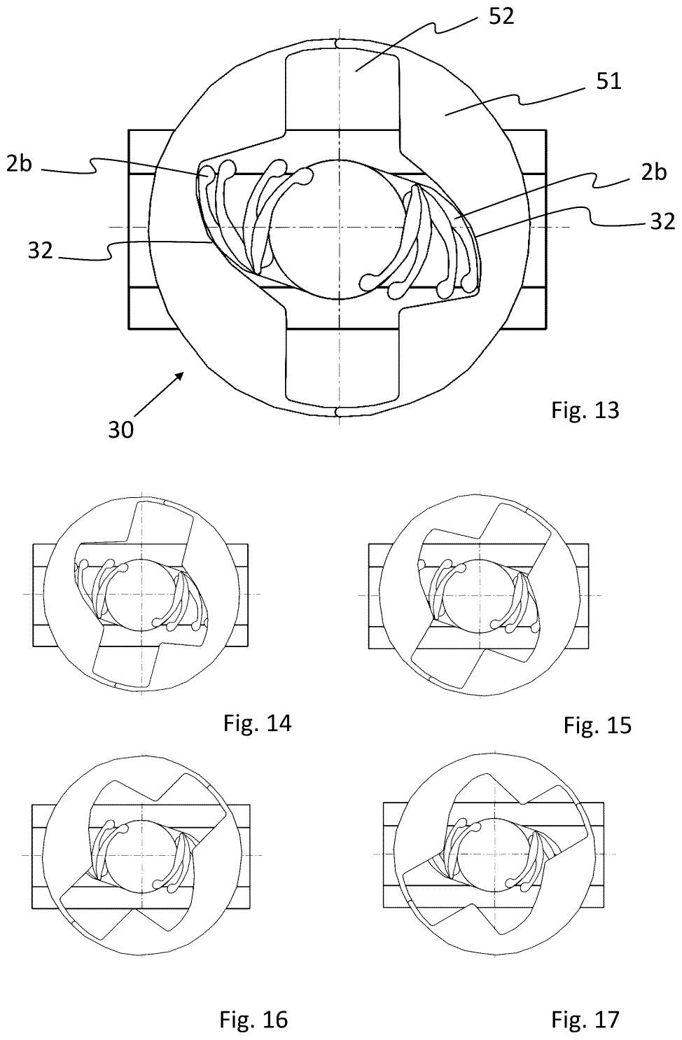

FIGS. 13 to 18 are schematic views of a cartridge 30 comprising a haptics slider in the form of a turntable 51.

The turntable 51 is mounted for rotation on the housing of the cartridge 30.

FIG. 13 is a plan view showing the non-activated state from above.

Turntable 51 has recesses 32 which have a curved shape.

In the non-activated state illustrated here, the arms of the haptics 2b can deflect into the recesses 32.

Furthermore, turntable 51 has a passage 52 for the folding member.

In the non-activated state illustrated here, the passage 52 for the folding member is aligned transversely to the folding member.

Since the recesses 32 do not provide enough space for the folding member, the folding member is inhibited from plunging into the housing of the cartridge in the non-activated state. Rather, it will abut on the turntable 51.

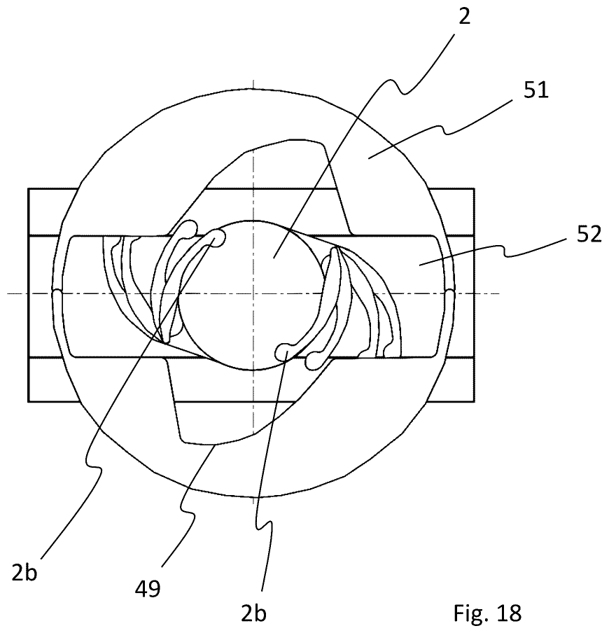

FIGS. 14 to 18 show how the user now turns the turntable 51 by a quarter turn until it reaches the final position shown in FIG. 18.

By virtue of the turning, the engagement surfaces 49 fold inward the arms of the haptics 2b.

In the final position, passage 52 is now aligned in the longitudinal direction and the folding member can now plunge through the passage 52, fold the lens 2 and transfer it into the passage below, which leads to the cannula.

Preferably, the turntable 51 is secured against being turned back, at least in its final position (for example by a snap-fit).

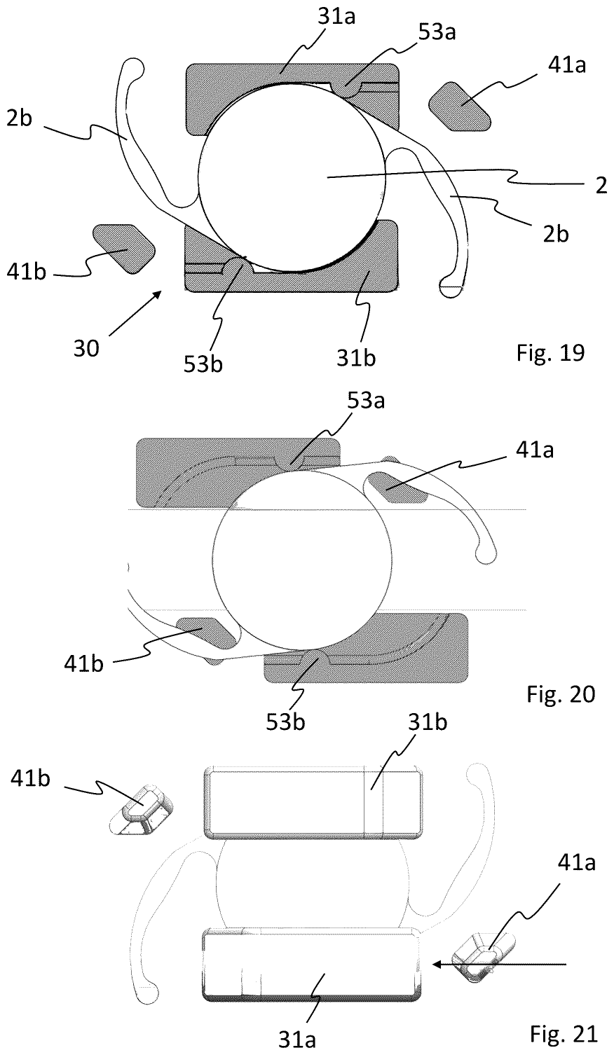

FIGS. 19 to 21 schematically show an embodiment of an injector in which the haptics slider can be arranged in a plane below the plane in which the lens 2 is carried.

FIG. 19 and FIG. 20 are longitudinal sectional views along a horizontal plane, with FIG. 19 showing the non-activated state of the haptics slider and FIG. 20 showing the activated state.

The haptics slider comprises two laterally opposite sliding members 31a, 31b which are movable relative to each other in the longitudinal direction.

In this embodiment, only the first sliding member 31a is movable. This simplifies the design of the injector.

In order to fold inward the haptics, the sliding members 31a, 31b each comprise a head 41a, 41b, similar to the exemplary embodiment according to FIGS. 5 to 7. In this exemplary embodiment, since only the first sliding member 31a is movable, the lens 2 has to be moved towards the head 41b in order to fold inward the arms of the haptics 2b.

For this purpose, the sliding members 31a, 31b each have a driver 53a, 53b.

The driver 53a, 53b engages laterally on the optical portion of the lens 2.

When now the sliding member 31a is moved thereby moving the driver 53a along the lens 2, the driver 53a will press the lens against the driver 53b. Lens 2 is resilient so that the drivers 53a, 53b can be pressed into the lens 2.

The lens 2 will now be rotated by the drivers 53a, 53b by an angle that is predefined depending on the design and will thereby be moved into the final position as illustrated in FIG. 20.

The heads 41a, 41b of the sliding members 31a, 31b will thereby fold inward the lens haptics, the inward folding being partly caused by the rotation of the lens.

FIG. 21 is a plan view of the underside of the cartridge.

It can be seen that there is enough space between the sliding members 31a, 31b with heads 41a, 41b, so that the passage between the sliding members 31a, 31b is sufficiently wide to allow the folded lens to pass between the sliding members 31a, 31b to be transferred into the passage through which the lens can be applied.

The components of the injector not shown here are preferably configured in accordance with the preceding exemplary embodiments. In particular, the injector may comprise a folding member which is arranged on a flap that will be closed before the intraocular lens 2 is applied.

The invention made it possible to provide an injector for intraocular lenses that is easier to operate. More particularly, the activation of the sliding members of a haptics slider can be coupled and/or the folding member can be inhibited until the haptics slider has been activated.

### LIST OF REFERENCE NUMERALS

1 Injector
2 Intraocular lens
2a Lens body (optical portion)
2b Haptics
10 Housing
11 Cannula
12 Flap
13 Folding member
14 Bearing block
15 Film hinge
16 Passage
17 Locking hook (for rear housing part)
18 Locking hook (flap)
19 Rail
20 Connecting portion for rear housing part
30 Cartridge
31a First sliding member
31b Second sliding member (31a+31b=haptics slider)
32 Recess
33 Groove
34 Rocker
35a, 35b Bar
36 Axis of rotation
37 Hinge
38 Locking hook
39 Further hinge
40 Rail
41a, 41b Head of sliding member
42 Support plane
43 Rail
44 Intermediate part
45 Passage
46 Rail
47 Front portion
48 Plunger
49 Engagement surface
50 Locking hook
51 Turntable
52 Passage
53a, 53b Driver
60 Container
61 Passage
62 Stop

1-16. (canceled)

17. A system, comprising:
a container; and
an injector for an intraocular lens, the injector comprising
a haptics slider for folding inward haptics of the intraocular lens, wherein the haptics slider can be activated by insertion into the container.

18. The system as claimed in claim 17,
wherein the injector comprises a cartridge for the intraocular lens that can be slidably fitted onto the injector, and
wherein the cartridge is insertable into the container and comprises the haptics slider.

19. The system as claimed in claim 17,
wherein the container is a heating container for heating the intraocular lens.

20. The system as claimed in claim 17,
wherein the container is a liquid-filled container for storing and transporting a hydrophilic intraocular lens.

21. The system as claimed in claim 18,
wherein the haptics slider can be activated by inserting the cartridge together with the injector into the container.

22. The system as claimed in claim 18,
wherein the cartridge and/or a housing of the injector can be pressed against a stop in order to activate the haptics slider.

23. An injector for an intraocular lens, comprising:
a housing with a plunger that is slideable inside the housing for applying the intraocular lens,
wherein the injector comprises a cartridge for the intraocular lens, which cartridge can be slideably fitted onto the housing of the injector, and
wherein the injector comprises a haptics slider which can be activated by the slideable fitting of the cartridge.

24. The injector as in claim 23,
wherein the haptics slider forms part of the cartridge.

25. An injector for an intraocular lens, comprising:
a housing with a plunger that is slideable inside the housing for applying the intraocular lens,
wherein the injector comprises a cartridge for the intraocular lens,
wherein the cartridge can be slideably fitted onto the housing of the injector,
wherein the injector has a folding member for folding the intraocular lens and a haptics slider for folding inward haptics of the intraocular lens, and
wherein
the folding member is only enabled for folding the intraocular lens after the haptics slider has been actuated, or
the haptics slider comprises first and second sliding members, the first and second sliding members being configured so as to move in the same direction while folding inward the haptics, or
the haptics slider comprises first and second sliding members, the first and second sliding members being movable towards each other for folding inward the haptics, or
the haptics slider is arranged on an underside of the cartridge, or
the haptics slider can be activated by connecting the cartridge, which is placed in a container, by connecting the cartridge with the injector.

26. The injector as in claim 25,
wherein the haptics slider is configured so as to be rotatable in form of a turntable.

27. The injector as in claim 26,
wherein in a folded position of the haptics, the haptics slider provides a passage for the folding member.

28. The injector as in claim 25, wherein the sliding members are arranged laterally next to
the intraocular lens or distal and proximal of the intraocular lens.

29. The injector as claimed in claim 25,
wherein the haptics slider is arranged in a plane above or below a support area for the intraocular lens.
